(19) United States
(12) Patent Application Publication
(10) Pub. No.: US 2011/0178549 A1
(43) Pub. Date: Jul. 21, 2011

(54) Acellular dermal surgical sutura

(76) Inventor: Zsuzsanna Juhasz, Budapest (HU)

(21) Appl. No.: 12/925,553

(22) Filed: Oct. 25, 2010

Related U.S. Application Data
(60) Provisional application No. 61/336,356, filed on Jan. 21, 2010.

(51) Int. Cl. A61L 17/08

(52) U.S. Cl. 606/229

(57) ABSTRACT

The invention relates to a surgical suture formed from an acellular, collagen based tissue material. After healing, the suture material will not absorb into the host, but will act as a structural template that will repopulate with viable cells from the host.

### CLAIM TO PRIORITY OF PROVISIONAL APPLICATION

This application claims priority under 35 U.S.C. §119(e)(1) of provisional application No. 61/336356, filed Jan. 21, 2010.

### TECHNICAL FIELD OF THE INVENTION

The technical field of this invention is surgical sutures, more specifically surgical sutures comprising of acellular tissue matrices.

### BACKGROUND OF THE INVENTION

Surgical wounds and incisions are typically closed after surgery by suturing. The materials commonly used for the suture fall into two primary categories—absorbable and non absorbable.

Non-absorbable sutures may be made of silk or synthetic materials such as polypropylene, polyester or nylon. Stainless steel wires are commonly used in orthopedic surgery and for sternal closure in cardiac surgery. These may or may not have coatings to enhance their performance characteristics. Non-absorbable sutures are used either on skin wound closure, where the sutures can be removed after a few weeks, or in internal environments. Examples include the heart (with its constant pressure and movement) or the bladder (with adverse chemical conditions). Non-absorbable sutures may cause less scarring because they provoke less immune response, and thus may be used where cosmetic outcome is important. They must be removed after a certain time, or may be left permanently.

Absorbable suture materials include the original catgut as well as the newer synthetics such as polyglycolic acid, polylactic acid, polydioxanone, and caprolactone. They are broken down by various processes including hydrolysis and proteolytic enzymatic degradation. Depending on the material, the process can be from ten days to eight weeks duration. They are used in patients who cannot return for suture removal, or in internal body tissues. In both cases, they will hold the body tissues together long enough to allow the healing process to conclude, but will eventually disintegrate so that they do not leave foreign material or require further procedures. Absorbable sutures can cause inflammation and may be rejected by the body rather than absorbed.

### SUMMARY OF THE INVENTION

A new approach to suturing is taught in this application where the suturing material is comprising of an acellular tissue matrix.

The suture described in this invention neither has to be removed after healing, nor does it absorb or loose strength like the currently known suture materials. After use the matrix will function as a structural template which then repopulates with new viable cells from the host.

### DETAILED DESCRIPTION OF PREFERRED EMBODIMENTS

An acellular tissue matrix is primarily collagen based and is processed from biological tissues, primarily but not exclusively from human skin or dermis. As part of the processing steps which are well known in the art all viable cellular material is removed from the tissue, leaving in place the non cellular protein matrix comprising of collagen and other proteins devoid of viable cells. This matrix may then be partially or completely dehydrated, usually but not exclusively by freeze drying. This results in a stable material that may be stored, and then rehydrated before use.

One of the primary advantages of such a acellular matrix is the absence of biocompability problems and tissue rejection as the matrix will not be recognized as a foreign body.

One method of preparing said acellular tissue matrix is described in U.S. Pat. No. 5,336,616, but any number of other methods may be used that result in a stable matrix devoid of all viable cells.

Acellular tissue matrices are currently being used in surgery where relatively large areas must be enclosed and/or reinforced. An example of this would be hernia repair where the defect is covered with a reinforcing material that may be a synthetic mesh, or a layer of an acellular tissue matrix. Said reinforcing material is usually fixed in the desired location by suturing with one of the currently used suture materials.

The novel suturing material described herein is formed from an acellular tissue matrix by shaping the matrix into round, flat or rectangular filament-like shapes, with dimensions similar to the dimensions currently used in the manufacture of suture materials.

The filament shape may be developed by various methods known in the art, such as die cutting or slicing. The suturing needle may also be attached to the suture by any of the currently known methods such as crimping.

What is claimed is:

1. A surgical suture comprising of a collagen based tissue matrix devoid of viable cells.

2. The surgical suture of claim 1 wherein said matrix comprises skin.

3. The surgical suture of claim 1 wherein said matrix comprises dermis.

4. The surgical suture of claim 1 being prepared by forming a round, flat or rectangular filament from said tissue matrix.
